(19) United States
(12) Patent Application Publication
(10) Pub. No.: US 2011/0210056 A1
(43) Pub. Date: Sep. 1, 2011

(54) GAS PHASE APPROACH TO IN-SITU/EX-SITU FUNCTIONALIZATION OF POROUS GRAPHITIC CARBON VIA RADICAL-GENERATED MOLECULES

(75) Inventors: Matthew R. Linford, Orem, UT (US); David Scott Jensen, Provo, UT (US); Michael A. Vail, Genola, UT (US)

(73) Assignees: BRIGHAM YOUNG UNIVERSITY, Provo, UT (US); US SYNTHETIC CORPORATION, Orem, UT (US)

(21) Appl. No.: 13/035,597

(22) Filed: Feb. 25, 2011

Related U.S. Application Data
(60) Provisional application No. 61/339,091, filed on Feb. 26, 2010.

(51) Int. Cl. B01D 15/08; C07C 43/21; C07C 15/20; B05D 5/00; B05D 3/06

(52) U.S. Cl. 210/198.2; 568/300; 585/21; 427/244; 427/553

(57) ABSTRACT

Embodiments disclosed herein include graphitic stationary phase materials functionalized through a gas-phase functionalization reaction, as well as and methods for making and using these materials, including the use of these materials in separation technologies such as, but not limited to, chromatography and solid phase extraction. In an embodiment, a functionalized graphitic stationary phase material may be prepared from high surface area porous graphitic carbon and a radical forming volatilized functionalizing agent. The radical forming volatilized functionalizing agent produces an intermediate that forms a covalent bond with the surface of the porous graphitic material and imparts desired properties to the surface of the graphitic carbon.

### CROSS-REFERENCE TO RELATED APPLICATIONS

This application claims the benefit of U.S. Provisional Patent Application No. 61/339,091, entitled “GAS PHASE APPROACH TO IN SITU/EX SITU FUNCTIONALIZATION OF POROUS GRAPHITE CARBON VIA RADICAL-GENERATED MOLECULES,” filed 26 Feb. 2010, which is incorporated herein, in its entirety, by this reference.

### BACKGROUND

Chromatography and solid-phase extraction (“SPE”) are commonly-used separation techniques employed in a variety of analytical chemistry and biochemistry environments. Chromatography and SPE are often used for separation, extraction, and analysis of various constituents, or fractions, of a sample of interest. Chromatography and SPE may also be used for the preparation, purification, concentration, and clean-up of samples.

Chromatography and solid phase extraction relate to any of a variety of techniques used to separate complex mixtures based on differential affinities of components of a sample carried by a mobile phase with which the sample flows, and a stationary phase through which the sample passes. Typically, chromatography and solid phase extraction involve the use of a stationary phase that includes an adsorbent packed into a cartridge or column. A commonly-used stationary phase includes a silica-gel-based sorbent material.

Mobile phases are often solvent-based liquids, although gas chromatography typically employs a gaseous mobile phase. Liquid mobile phases may vary significantly in their compositions depending on various characteristics of the sample being analyzed and on the various components sought to be extracted and/or analyzed in the sample. For example, liquid mobile phases may vary significantly in pH and solvent properties. Additionally, liquid mobile phases may vary in their compositions depending on the characteristics of the stationary phase that is being employed. Often, several different mobile phases are employed during a given chromatography or SPE procedure. Stationary phase materials may also exhibit poor stability characteristics in the presence of various mobile phase compositions and/or complex mixtures for which separation is desired. The poor stability characteristics of stationary phase materials in some mobile phases and complex mixtures, in some cases, may even preclude the possibility of using chromatography or solid phase extraction to perform the desired separation.

High surface area porous graphitic carbon, also referred to herein as “HSAPGC” and “porous graphitic carbon,” has many unique properties such as chemical and thermal stability, thermal conductivity, and polarizability, which makes it useful for liquid chromatography. Since the surface of graphite is polarizable, the retention mechanism of porous graphitic carbon is a charge-induced interaction between itself and other polar analytes.

### SUMMARY

Embodiments disclosed herein include functionalized graphitic stationary phase materials and methods for making and using these materials, including the use of these materials in separation technologies such as, but not limited to, chromatography and solid phase extraction. In an embodiment, a functionalized graphitic stationary phase material may be prepared from high surface area porous graphitic carbon and a radical forming gas-phase functionalizing agent. Use of a gas-phase, rather than a liquid phase approach, may provide the functionalized material with increased retention times and less tailing of the chromatographic peaks as compared to liquid phase functionalization. The radical forming functionalizing agent produces an intermediate that forms a covalent bond with the surface of the porous graphitic material and imparts desired properties to the surface of the graphitic carbon. For example, a plurality of alkyl-group-containing functional group molecules (e.g., tert-butyl and/or amyl groups) may be covalently bonded to the surface of the porous graphitic carbon. The functionalized graphitic stationary phase material may advantageously exhibit unique selectivity and good thermal and chemical stability.

In one embodiment, a method for preparing a functionalized graphitic stationary phase material includes providing a high surface area porous graphitic carbon having a porosity and surface area suitable for use as a stationary phase. The method also includes providing a gas-phase functionalizing agent capable of forming a radical that may form a covalent bond with graphitic carbon. The gas-phase functionalizing agent is caused to form a radical intermediate and reacted with the porous graphitic carbon. The functionalizing agent may be provided in the gas-phase by heating the functionalizing agent and the porous graphitic carbon. In one embodiment, the functionalizing agent undergoes cleavage upon heating or exposure to a particular wavelength of light, leading to the formation of radical intermediate(s). The radical intermediate forms a covalent bond with the surface of the porous graphitic material, thereby yielding the functionalized graphitic stationary phase material.

The radical forming functionalizing agent may include one or more alkyl groups (e.g., a tert-butyl group and/or an amyl group) and optionally one or more heteroatoms (e.g., N and/or O). For example, in one embodiment, the radical forming agent may be an azo compound or a dialkyl peroxide. The step of forming the radical intermediate may be promoted using heat, light, chemicals, or combinations of the foregoing. In any case, the functionalizing agent is provided in a gas-phase rather than a liquid-phase, and the functionalization reaction typically occurs in a heated environment.

In another embodiment, a separation apparatus for performing chromatography or solid phase separation is described. The separation apparatus includes a vessel having an inlet and an outlet. Any of the functionalized graphitic stationary phase materials disclosed herein may be disposed within the vessel. The vessel may be a column or a cassette suitable for use in the fields of chromatography and/or solid phase separation (e.g., high performance liquid chromatography (“HPLC”) or ultra performance liquid chromatography (“UPLC”)).

The separation apparatus may be used to physically separate different components from one another. In one embodiment, a mobile phase including at least two different components to be separated is caused to flow through the functionalized graphitic stationary phase material to physically separate the at least two different components. At least one of the two different components is recovered.

The functionalized stationary phase material may be used in some embodiments with a mobile phase that would typically degrade commonly used stationary phase materials, such as a silica gel. For example, the mobile phase may include organic solvents, and/or highly acidic or highly basic solvents (e.g., pH greater than 10 or less than 2).

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a flow diagram of a method for preparing a functionalized graphitic stationary phase according to an embodiment;

FIG. 2 is a cross-sectional view of an embodiment of a separation apparatus including any of the functionalized graphitic stationary phase materials disclosed herein;

FIG. 3 is a principal component analysis (“PCA”) of time-of-flight secondary positive ion mass spectrometry spectra (“ToF-SIMS”) of a functionalized graphitic stationary phase material prepared according to Example 1; and

FIG. 4 is a PCA of time-of-flight secondary negative ion mass spectrometry spectra of a functionalized graphitic stationary phase material prepared according to Example 1.

### DETAILED DESCRIPTION

### I. INTRODUCTION

Embodiments disclosed herein are directed to functionalized graphitic stationary phase materials, methods for making such materials through a gas-phase functionalization of the graphitic material, and separation apparatuses (e.g., chromatography and solid-phase extraction apparatuses) and separation methods that employ such gas-phase functionalized graphitic stationary phases.

### II. COMPONENTS USED TO MAKE POROUS COMPOSITE PARTICULATE MATERIALS

Components useful for preparing the functionalized graphitic stationary phase material include, but are not limited to, high surface area porous graphitic carbon and radical forming functionalizing agents.

A. High Surface Area Porous Graphitic Carbon

The functionalized graphitic material may be prepared using a high surface area porous graphitic carbon. The high surface area porous graphitic carbon includes graphite, which is a three-dimensional hexagonal crystalline long range ordered carbon that may be detected by diffraction methods. In one embodiment the high surface area porous graphitic carbon is mostly graphite or even substantially all graphite. The surface of the porous graphitic carbon may include domains of hexagonally arranged sheets of carbon atoms that impart aromatic properties to the carbon. In other embodiments, the functionalized graphitic material may also include non-graphitic carbon (e.g., amorphous carbon) in addition to the high surface area graphitic carbon. The graphitic nature of the porous graphitic carbon provides chemical and thermal stability in the presence of traditionally harsh solvents such as organic solvents (e.g., methanol) and highly acidic or highly basic solvents.

The functionalized graphitic material exhibits an average particle size, porosity, and surface area suitable for use in separation techniques such as chromatography and solid phase separation. In an embodiment, the porous graphitic material may have an average particle size that is in a range from about 1 μm to about 500 μm, more specifically about 1 μm to about 200 μm, or even more specifically in a range from about 1 μm to about 100 μm. The desired average particle size may depend on the application in which the stationary phase is to be used. In one embodiment, the porous graphitic carbon particles have an average particle size in a range from about 1 μm to 10 μm, more specifically about 1.5 μm to about 7 μm. This range may be suitable for HPLC applications and the like. In another embodiment, the average particle size may be in a range from about 5 μm to about 500 μm, or more specifically in a range from about 10 μm to about 150 μm. This larger range may be suitable for solid phase extraction applications and the like.

The high surface area porous carbon may be prepared using any technique that provides the desired surface area, particle size, and graphitic content. In one embodiment, porous graphitic carbon may be prepared by impregnating a silica gel template with phenol-formaldehyde resin, followed by carbonization of the silica-resin composite, dissolution of the silica to form a porous carbon intermediate, and finally graphitization of the porous carbon intermediate to form porous graphitic carbon. This process produces a 2-dimensional crystalline surface of hexagonally arranged carbon atoms over at least some surfaces of the porous carbon intermediate. Its pore structure may be similar to that of the original silica template. The open pore structure may provide the porous graphitic carbon mass transfer properties comparable to those of silica gels but with superior structural integrity and resistance to chemical degradation.

B. Radical Forming Functionalizing Agents

The methods for preparing the functionalized graphitic stationary phase material include the use of a radical forming functionalizing agent. The radical forming functionalizing agent includes one or more alkyl groups and optionally one or more heteroatoms. When bonded to the surface of the porous graphitic carbon, the alkyl and heteroatoms bonded thereto impart properties that are desirable for separating components of a mobile phase. The functionalizing agent is selected to be capable of forming a radical intermediate that may react with and form a covalent bond with the graphitic surface of the high surface area porous graphitic carbon.

In one embodiment, the radical forming functionalizing agent forms a carbon radical intermediate that may form an sp³ hybridized bond with one of the hexagonally arranged carbon atoms in the graphitic surface of the porous graphitic carbon material.

Several types of radical forming compounds may be used as radical forming functionalizing agents. In one embodiment, the radical forming agent may be a compound typically used in polymerization reactions as an initiator. In some embodiments, the radical forming functionalizing agent may be a compound that decomposes to form one or more radical species. The decomposition of the radical forming agent may be caused by heat, light, and/or chemical activators.

The radical forming functionalizing agent is in a gas-phase such that the functionalization may be carried out within a gas-phase, rather than a liquid or in solution liquid phase. Such gas-phase functionalizing agents may typically be of relatively low molecular weight so as to be volatilized upon addition of heat and/or application of low pressure. According to one embodiment, the gas-phase functionalizing agent has a molecular weight of not more than about 500, more specifically not more than about 400, and more specifically not more than about 300.

Examples of compounds that may be used as radical forming functionalizing agents include, but are not limited to, alkyl halides, azo compounds, benzoyl peroxide, diacyl peroxides, alkyl peroxy acids, dialkyl peroxides, tri-peroxides, peroxyesters, perfluoronated peroxides, tertiary alcohols, hydroperoxides, molecules with two or more double bonds, epoxide groups, or molecules of the form CH₂═CHC(CH₃)₂OH, and similar compounds. These compounds may be used as neat compounds or solvated in an appropriate solvent. In other words, such molecules may be used as functionalizing agents for porous graphitic carbon either neat, in solution, or after vaporization.

Fluoronated compounds may provide monolayer functionality on the graphitic surface as fluorinated radicals may not easily abstract any attached fluorine atoms from the surface. Suitable azo compounds may include symmetrical azo compounds, asymmetrical azo compounds, and perfluoronated azo compounds (which may be symmetrical, asymmetrical, or hybrid organic/perfluoronated compounds). Specific azo compounds that may be suitable azobisisobutyronitrile (“AIBN”) or azo-tert butane (“ATB”). A specific hydroperoxide may include (CH₃)₃COOH.

Exemplary alkyl halides may include tertiary alkyl halides, of the form R₁R₂R₃CX, where X is a halogen, particularly bromine or iodine. Upon heating, these species would generate tertiary carbon radicals that would be expected to covalently bond to the porous graphitic carbon material. Other radical producing species that may be suitable may include perfluoroazooctane, fluoroalkyl iodides, fluorodiacyl peroxides, and other diacyl peroxides.

An exemplary diperoxide or triperoxide used to functionalize porous graphitic carbon and/or cross link with another radical forming functionalizing agent may include a compound having the structure:

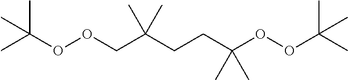

A specific dialkyl peroxide that may be suitable is di-tert-amyl peroxide (“DTAP”), which is a tertiary peroxide. Peroxides that do not have tertiary oxygen atoms may also be suitable. Other suitable dialkyl peroxides may include alkyl groups having longer chains (e.g., between about 10 and about 30 carbons, between about 12 and about 24 carbons, e.g., 18 carbons).

In another embodiment, diols of the form HOC(CH₃)₂(CH₂)ₙC(CH₃)₂OH or HOC(CH₃)₂C₆H₄C(CH₃)₂OH may act as cross linking reagents for the covalently bonded thin films and/or add functionality to the final films in the form of —OH groups. One particular contemplated diol that could be used with a tertiary peroxide such as DTAP would be that corresponding to the diperoxide:

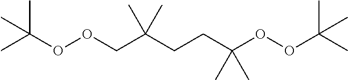

In another embodiment, one may add a molecule to the reaction that would react with an oxygen-centered radical (e.g., DTAP) or radical at a surface. Possible species might include molecules that contain one or more carbon-carbon double bonds, e.g., acrylate groups (acrylic acid, methyl acrylate, butyl acrylate, etc.), methacrylate groups (methacrylic acid, methyl methacrylate, dodecyl methacrylate, etc.), vinyl ether groups, acrylamide groups, styrenic molecules (e.g., styrene (CH₂CHC₆H₅), divinylbenzene (CH₂CHC₆H₄CHCH₂), 4-methylstyrene, 4-trifluoromethylstyrene), butadiene, isoprene, or combinations thereof. The quantity of such a reagent might be low enough to prevent a significant amount of polymerization, but large enough to add functionality to the stationary phase. Alternatively, some polymerization may occur in solution or gas-phase and this polymer would be washed away after surface functionalization. Under some circumstances, it may also be advantageous to have some selective adsorption of a polymer to a surface.

In many embodiments, it would be advantageous to degas the reagent before introducing it into the column (or onto the particles) for surface functionalization.

In one embodiment, the radical forming functionalizing agent may be a “Vazo free” radical source sold by DuPont (USA). The DuPont Vazo® free radical sources are substituted azonitrile compounds that thermally decompose to generate two free radicals per molecule and evolve gaseous nitrogen. The rate of decomposition is first-order and is unaffected by the presence of metal ions.

In one embodiment, an alcohol may be mixed with a dialkyl peroxide such as DTAP, with the expectation of the following hydrogen atom transfer:

CH₃CH₂C(CH₃)₂O.+ROH→CH₃CH₂C(CH₃)₂OH+RO.

In this manner, a different oxygen-centered radical may be generated in situ, which would also be expected to add to the porous graphitic carbon and/or previously adsorbed alkyl groups. Various possible alcohols may be used for this purpose. One or more of the R groups in the tertiary alcohol might be aliphatic, aromatic, or contain some other desired functionality, e.g., be fluorinated, have a carboxyl group, an ether linkage, etc. More particularly, R may be a phenyl group, a benzyl group, a naphthyl group, a biphenyl group, an alkyl chain that contains 18 carbons, an alkyl chain that contains 8 carbons, an alkyl chain that contains 4 carbons, a perfluorinated alkyl chain, etc.

Such alcohols, as well as DTAP include an oxygen heteroatom. It is believed that the tertiary position of the oxygen atom on a DTAP radical fragment may be important because it has no alpha hydrogen. For example, species of the type: RCH₂CH₂CH₂O. may be particularly susceptible to hydrogen abstraction by another radical to create the following aldehyde: RCH₂CH₂CH═O. Further hydrogen abstractions may result in increasingly conjugated systems, e.g., RCH═CHCH═O, some of which might even show some tendency to polymerize, or to act as radical traps. Extensive polymerization may lead to plugging of a portion of the pores within the porous graphitic carbon, and thus in at least some embodiments, such species may be less preferred.

Tertiary alcohols may be synthesized by any suitable method. For example, a one step route to such compounds may be possible from an alkyl Grignard or lithium reagent and acetone, where the reaction below assumes an aqueous, mildly acidic workup:

RMgX+CH₃C(O)CH₃→RC(CH₃)₂OH

or

RLi+CH₃C(O)CH₃→RC(CH₃)₂OH

Another possible synthesis is a Markovnikov addition to double bonds under acidic conditions (usually bubbling HX gas through a solution of the alkene), followed by reaction with water:

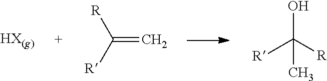

Alternatively and perhaps more directly, the tertiary alcohol may be synthesized via acid catalyzed hydration of an alkene using H₂SO₄. Various other mechanisms for synthesizing a tertiary alcohol will be apparent to one of skill in the art in light of the present disclosure.

In the case where the functionalizing agent includes one or more heteroatoms, the heteroatoms may be bonded to an alkyl group. The alkyl group may be substituted or unsubstituted straight chain, branched or cyclic alkyl groups. In one embodiment, the alkyl group may include a ring structure with aromaticity. The one or more heteroatoms may be one or more halides.

In some cases the functionalizing agent may be a halogen-substituted or polyhalogen-substituted alkane or benzene. In one embodiment, the halogen substituted compound is a fluorinated alkyl compound. Examples of halogen-substituted alkyl compounds include perfluorinated substituents or compounds with the formula RfX where Rf is a fluorinated alkyl group and X is chlorine, bromine, or iodine. A more specific, but non-limiting example of a perfluorinated alkyl compound is heptadecafluoro-1-iodooctane. Thermolysis of the X component of RfX produces an Rf radical that can create an sp³ bond with the porous graphitic carbon.

Another example of a perfluoro alkyl compound that may be used is a polyfluorobenzene compound. In this case, the Rf moiety includes a benzene ring. A more specific, non-limiting example of a polyfluorobenzene compound that may be used is pentafluoroiodobenzene.

In another embodiment, the functionalizing agent may be a perfluoronate compound (RfCOO-M+). At elevated temperatures the RfCOO-M+ compound undergoes decarboxylation which produces CO₂ and radical Rf species. The radical species reacts with the porous graphitic carbon to produce an sp³ linkage between the graphite and Rf molecule.

In yet another embodiment, the functionalizing agent may be a perfluorinated azo compound (RfN₂). Thermolysis of the carbon-nitrogen bond occurs at elevated temperatures, which produces N₂ and Rf radicals. The resulting Rf radicals react with the porous graphitic carbon to produce sp³ linkages between the graphite and the Rf molecules.

The radical producing functionalizing agent may be caused to form a radical using heat, light, chemical agents, or a combination of the foregoing. For example, As an alternative to thermally induced cleavage, many suitable functionalizing agent compounds undergo radical producing cleavage upon exposure to light of a specific wavelength (e.g., UV). Where light of a particular wavelength is used as the degrading trigger, the modifying agent may be volatilized and allowed to pass through an optically transparent material via an inert carrier gas. The molecule is cleaved (e.g., at the heteroatom bond) by the specific wavelength needed to create the reactive radical species. The carrier gas may carry the produced radical into the porous graphitic carbon material where the functionalization reaction occurs. Such a process may be performed in-situ (e.g., within a prepacked column) or ex-situ. The pressure of the carrier gas may be increased or decreased as desired to cause the reaction to occur in a desired area of the column or ex-situ reaction container.

In a specific embodiment, the temperature at which a radical forms is at least about 150° C. and more specifically at least about 200° C. Similarly, the functionalizing agent is volatilized (i.e., in a gas-phase) at the applied temperature. Alternatively, low pressure, or a combination of low pressure and heating may be employed to volatize the functionalizing agent. Generally, the temperature at which radical formation occurs, the wavelength that causes radical formation, and/or the chemicals that cause radical formation may be specific to the particular radical forming compound.

### III. METHODS FOR GAS-PHASE FUNCTIONALIZATION OF GRAPHITIC STATIONARY PHASE

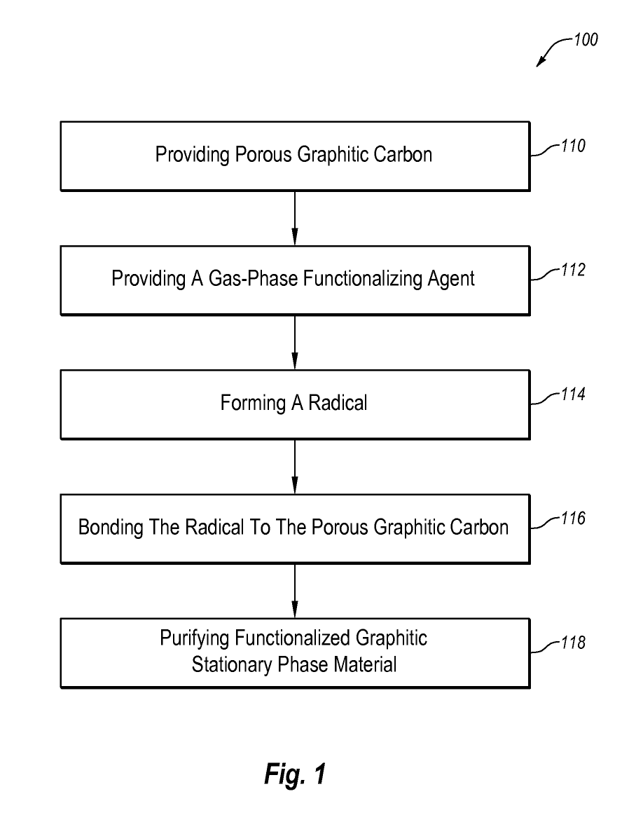

Reference is now made to FIG. 1 which illustrates a flow diagram 100 of an embodiment of a method for making functionalized graphitic stationary phase materials. At 110 and 112, a porous graphitic carbon and a radical forming agent are provided, respectively. The porous graphitic carbon and radical forming agent may be any of those described above or compounds that provide a similar functionality as the materials mentioned herein.

At 114, a radical intermediate is formed from the radical forming gas-phase functionalizing agent. The particular method in which the radical may be formed depends on the nature of the particular functionalizing agent. Functionalizing agents suitable for use in the methods described herein may be activated by heat, light, chemical activators, or combinations of the foregoing. In many cases, the functionalizing agent decomposes in the presence of the heat, light, and/or a chemical activator and/or undergoes a change involving cleavage resulting in formation of the radical. The decomposition typically produces a reactive radical intermediate suitable for covalently bonding with the graphitic surface and may produce a non-functionalizing radical that then forms a non-reactive species. Examples of relatively non-reactive species that may form during the reaction include, but are not limited to, nitrogen gas, carbon dioxide gas, and metal halides.

In one embodiment, an activating agent may be used in combination with the functionalizing agent to promote formation of the radical intermediate. In one embodiment, the activating agent may include a metal such as, but not limited to, group IB metals including copper, silver, gold, and combinations thereof. Metal activating agents may be used in combination with polyfluoro-alkyl compounds to form radicals. In one non-limiting example, a IB metal such as copper may be used with a fluorinated alkyl compound such as, but not limited to, pentafluoroiodobenzene to enhance perfluoroalkylation. The IB metal may also act as a scavenger of undesired radicals. The reaction scheme below is currently believed to be the route of perfluorination with pentafluoroiodobenzene and copper:

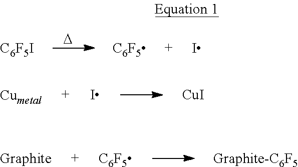

In one embodiment, the use of heat to form a radical may be beneficial as the heat may also aid in volatilizing the functionalizing agent so that the reaction occurs in a gas-phase. Furthermore, application of heat may aid in ensuring a relatively even distribution of the formed radical within the pores of the porous graphitic carbon. Even distribution of the functionalization of the porous graphitic carbon may help achieve high separation efficiency in chromatography and solid phase extraction procedures using the functionalized graphitic material.

In one embodiment, the formation of the radical intermediate may be carried out at a temperature of at least about 150° C., more specifically at least about 200° C. In one embodiment, the radical intermediate is formed at a temperature in a range from about 150° C. to about 500° C., more specifically in a range from about 200° C. to about 300° C. In any case, the temperature and/or pressure is such as to volatilize the functionalizing agent, and in the case of thermally induced cleavage, the temperature is also sufficient to induce cleavage. For example, DTAP has a boiling point of about 146° C. at atmospheric pressure, while ATB has a boiling point of about 48° C. under a reduced pressure of about 8 mm Hg. At least some suitable functionalizing agents (e.g., DTAP and ATB) will undergo hemolytic cleavage. Other temperatures may be used so long as the temperature is sufficient to cause thermolysis of the radical producing functionalizing agent, if thermal induction is the mechanism of cleavage.

In the case where the radical producing functionalizing agent is a light activated compound, the intermediate may be formed by exposing the functionalizing agent to the particular wavelength that causes photolysis of the functionalizing agent. The particular wavelength that induces radical formation is generally specific to the particular functionalizing agent. In many cases, the photolysis wavelength is within the UV portion of the spectrum. In the case of many functionalizing compounds, the thermally or photo induced cleavage occurs at heteroatom bonds (e.g., C—N bonds or C—O bonds).

In one embodiment, the reaction may be carried out in an inert environment. For example, the reaction mixture and/or chamber may be purged with argon, nitrogen, or another suitable inert gas to remove oxygen. Removing oxygen from the reaction mixture and/or reaction chamber advantageously minimizes the formation of oxygen functional groups on the surface of the graphite (e.g., minimizes formation of hydroxyl and carboxyl groups). The reaction vessel may also be maintained under vacuum to evacuate undesired reactive species. The use of reduced pressure conditions (e.g., vacuum) may also aid in volatilizing the functionalizing agent.

The reaction may be carried out in-situ or ex-situ. For example, the functionalizing reaction may be carried out with the porous graphitic carbon disposed within a column or other separation container, while the functionalizing agent is introduced into the column in order to decompose, forming the desired radicals, which then covalently bond to the porous graphitic carbon. Alternatively, the reaction may be carried out ex-situ through a similar gas-phase approach, but the graphitic material may be placed within a continuously agitated tumbler where the functionalizing agent may be continuously introduced or introduced at desired repeated intervals. The tumbler or other reaction container may be contained within an oven at elevated temperature. Where a continuous tumbler is not used, the graphitic material may be shaken or otherwise agitated at repeated intervals to provide for a more homogenous coating of the functionalizing coating on the graphitic particles.

In another embodiment that may be particularly suitable for in-situ functionalization a zone heater may be used to thermally degrade the gas-phase functionalizing agent in a particular zone of the column or other separation device. Such an embodiment may provide the ability to control the degree of functionalization at any particular location or zone within the column. A continuous or discontinuous flow of the functionalizing agent may be forced through the column with an inert carrier gas (e.g., argon or nitrogen). The heated zone may be set at a temperature that causes the volatilized functionalizing agent to thermally degrade. When the extent of functionalization is achieved, the heated zone may be moved down the column to functionalize the graphitic carbon material within another zone. Functionalization of the graphitic carbon material may be repeated to result in covalent bonding of additional alkyl functional groups to the graphite, if desired.

At 116 of method 100, the radical intermediate reacts with the porous graphitic carbon. This step is generally carried out by mixing the radical intermediate with the porous graphitic carbon. In one embodiment, the stoichiometric amount of functionalizing agent molecules per carbon atom in the porous graphitic carbon may be at least about 3 (i.e., a ratio of about 3:1), more specifically at least about 4 (i.e., a ratio of about 4:1).

The radical intermediates are highly reactive and form a covalent bond with the carbon in the graphitic sheet on the surface of the porous graphitic carbon. The formation of the covalent bond consumes the radical intermediate and yields the functionalized graphitic stationary phase material. The reaction components are allowed to react for a sufficient time to obtain the desired functionalization at a desired yield. The concentration of the functionalizing agent and the duration of the reaction determine the extent of functionalization. Because the functionalizing agent is volatilized in the gas-phase, and because of the relatively elevated temperature, the reaction may proceed more quickly than if the functionalization were carried out within a liquid phase. For example, in one embodiment, the functionalization step (including introduction of the functionalization agent) is allowed to proceed for between about 30 minutes and about 4 hours, more specifically between about 30 minutes and about 2 hours, not more than about 2 hours, or even more specifically between about 1 hour and about 2 hours. Introduction of the functionalizing agent may be continuous, or non-continuous, provided in aliquots at repeated intervals.

The radical intermediate is typically formed from the gas-phase volatilized functionalizing agent in the presence of the graphitic porous carbon due to the ephemeral nature of radicals. For example, the functionalizing agent may be introduced into a furnace (e.g., a tube furnace) with the porous graphitic carbon and then heated to volatilize the functionalizing agent and form the radical intermediate. Alternatively, heating to volatilize the functionalizing agent may be performed prior to introduction into the furnace with the porous graphitic carbon, followed by further heating to cleave the functionalizing agent. Of course, forming the radical in the presence of the porous graphitic carbon is not required so long as the radical intermediate lasts long enough to react with the porous graphitic carbon once the two materials are brought into contact.

Reaction at 116 may be carried out in an inert environment to prevent oxygen from reacting with the carbon in the porous graphitic carbon. This may be particularly important in reactions where the temperature is elevated. Oxygen may be removed from the reaction mixture by purging the reaction vessel with an inert gas such as, but not limited to, argon, nitrogen, or combinations thereof.

In one embodiment, the radical producing agent may form a start site on the graphite where polymerization may occur. In one embodiment, the surface of the porous graphitic carbon may be further functionalized by hydrogen reduction. The graphitic material may be exposed to a hydrogen plasma to hydrogen terminate the carbon (i.e., to create C—H bonds in the graphitic material), to a water plasma to introduce hydroxyl moieties onto the graphitic material, to a chlorine plasma, or combinations of the foregoing. Further methods include creating an initiation site for atom transfer radical polymerization, which may form on a graphite edge or face. ATRP or another type of living polymerization may be allowed to proceed from this site to produce covalently bonded functional groups on the surface of the porous graphitic carbon. Polymers covalently bonded to the porous graphitic carbon may also be cross-linked using known methods.

At 118, the functionalized graphitic stationary phase material may be purified, if needed. Any purification 118 may include collecting the reaction product and heating the reaction product in a vacuum to evaporate non-bonded reagents such as, but not limited to, residual radical forming functionalizing agent. In one embodiment, the functionalized graphitic stationary phase may be heated at a temperature of at least about 60° C., more specifically at least about 70° C. for at least about 2 hours, more specifically at least about 12 hours, and even more specifically at least about 24 hours. The reaction product may also be cleaned using one or more solvents. For example, the functionalized graphitic stationary phase material may be cleaned with xylenes, a mixture of xylenes and hexanes (e.g., 1:1 ratio), methanol, or combinations thereof. In another embodiment, cleaning may be by Soxhlet extraction with perfluorohexane. Such Soxhlet extraction cleaning with a solvent may be carried out for at least 2 hours, more specifically at least 12 hours, and even more specifically at least 24 hours.

### IV. FUNCTIONALIZED GRAPHITIC STATIONARY PHASE

The functionalized graphitic stationary phase materials described herein provide desired sizes, porosity, surface areas, and chemical stability suitable for chromatography and solid phase extraction techniques. When used in chromatography and solid phase extraction, high-resolution separation may be achieved with relatively low back pressure.

The functionalized graphitic stationary phase materials may be provided in the form of finely divided discrete particles (e.g., a powder). Alternatively, the functionalized graphitic stationary phase materials may be provided as a monolithic structure having a porosity and surface area that is similar to finely divided discrete particles. When the functionalized graphitic stationary phase materials are provided as a monolithic structure, the body may exhibit dimensions suitable for use in a separation apparatus, such as, but not limited to, separation devices used in HPLC.

In one embodiment, the functionalized graphitic stationary phase material includes a plurality of graphitic particles having an average particle size in a range from about 1 μm to 500 μm, more specifically about 1 μm to 200 μm, or even more specifically in a range from about 1 μm to about 150 μm. In one embodiment, the functionalized graphitic stationary phase materials have an average particle size in a range from about 1 μm to about 10 μm, or more specifically about 1.5 μm to about 7 μm. This particle size range may be particularly useful for HPLC applications and the like. In another embodiment, the functionalized graphitic stationary phase materials may have an average particle size in a range from about 5 μm to about 500 μm, or more specifically in a range from about 10 μm to about 150 μm. This larger average particle size range may be more suitable for use in solid phase extraction applications and the like.

The functionalized graphitic stationary phase materials may include a desired surface area. The surface area per unit weight of the functionalized graphitic stationary phase materials depends to a large extent on the surface area of the porous graphitic carbon used to prepare the functionalized graphitic stationary phase materials. In one embodiment, the surface area per volume or surface area per mass characteristics of the graphitic stationary phase is substantially unchanged relative to the characteristics prior to functionalization (i.e., the surface area characteristics remain substantially the same). In an embodiment, the surface area per unit weight may be measured using the Brunauer Emmett and Teller (“BET”) technique and is in a range from 1-500 m²/g for functionalized graphitic stationary phase materials having a particle size in a range from about 1 μm to 500 μm, more specifically in a range from 25-300 m²/g, or even more specifically 50-200 m²/g. In one embodiment, the functionalized graphitic stationary phase materials have a particle size in a range from about 1 μm to 10 μm and may have a surface area per unit weight in a range from about 10-500 m²/g, more specifically in a range from 25-200 m²/g, and even more specifically in a range from 25-60 m²/g. In another embodiment, functionalized graphitic stationary phase materials having a particle size from about 10 μm to 150 μm may have a surface area per unit weight in a range from about 5-200 m²/g, or more specifically 10-100 m²/g. In yet another embodiment, functionalized graphitic stationary phase materials having an average particle size in a range from about 250 μm to about 500 μm may have a surface area per unit weight of at least about 5 m²/g, and even more specifically at least about 10 m²/g for functionalized graphitic stationary phase materials with an average particle size in a range from about 250 μm to about 500 μm.

The surface of the functionalized graphitic stationary phase materials differs from porous graphitic carbon in significant ways. The functionalized graphitic stationary phases described herein include alkyl functional groups that are bonded (e.g., covalently bonded) to the graphitic carbon. For example, the surface of the graphitic carbon may include substantially only graphene or may be partially graphene, with the alkyl groups extending away from the graphene at an angle to the surface of the graphitic carbon. For example, the angle at which the alkyl groups extend away from the graphene may be substantially perpendicular.

The functional groups provide physical differences in the molecular structure of the surface of the porous graphitic carbon and may have a significant impact on separation efficiencies. In addition, the one or more alkyl groups and optional heteroatoms may provide unique electrical properties that cause the surface to interact with solvents and solutes differently than a pure graphitic surface. Because the functional groups are covalently bonded, the functional groups are capable of withstand relatively harsh conditions, thereby avoiding leaching or undesired reactions with solvents and/or solutes. These differences allow the functionalized stationary phases described herein to be used as a stationary phase for separating materials that cannot be separated with pure porous graphitic carbon. In various embodiments, the amount of the surface area of the porous graphitic carbon that is covalently bonded with the alkyl functional groups may be about 10 percent to about 98 percent, about 25 percent to about 95 percent, about 50 percent to about 90 percent, or about 75 percent to about 98 percent.

The particular properties that the covalently bonded functional groups impart to the functionalized graphitic stationary phase material may depend on the particular functional groups bonded thereto. In one embodiment, the functional groups bonded to the graphitic carbon may be similar to the radical producing agent molecules described above, but may differ with respect to the radical producing moiety. For example, the radical forming agent may lose a halogen radical, nitrogen radical, or carbon radical in the formation of the radical intermediate. Thus, the functional groups bonded to the graphitic carbon may include the one or more alkyl groups and optionally one or more heteroatoms from the radical producing functionalizing agent molecules, but not the radical forming moiety. In other words, the covalently bonded functional group may typically be relatively stable so as to not be thereafter cleavable to form additional radicals. For example, in the case of DTAP, the covalently bonded functional group comprises a tert-amyl group (C₅H₁₁) and an oxygen heteroatom that forms the covalently bonded bridge between the amyl group and the graphitic material. In the case of ATB, the alkyl group comprises a tert-butyl group (C₄H₉), while no heteroatom is present, but the terminal carbon of the tert-butyl group becomes covalently bonded directly to the graphitic material.

In one embodiment, the functional groups may include alkyl groups having two or more carbons, more specifically 4 or more carbons, and even more specifically 6 or more carbons. The alkyl groups may include ring structures of 4 or more atoms, more specifically 6 or more atoms. In one embodiment, the ring structures may be aromatic. In one embodiment, the functional group may be an alkyl halide. Examples of alkyl halides that may be exhibited on the surface of the graphitic carbon include, but are not limited to, perfluoroalkyl groups and polyfluorobenzene groups. More specifically, the alkyl halide may include a heptadecafluoro octane group and/or a pentafluorobenzene group.

In one embodiment, the alkyl group may comprise a C₁₈ alkyl group.

The extent of functionalization (i.e., the number of functionalizing agent molecules on the graphitic surface) is at least sufficient to cause an appreciable difference in the separation characteristics of the functionalized graphitic stationary phase as compared to non-functionalized porous graphitic carbon. In one embodiment, the extent of functionalization may be measured according to the atomic weight percent of the atoms in the functional group as a total atomic weight percent of the stationary phase material. In one embodiment, the atomic weight percent of the functional groups is at least about 1 atom %, more specifically at least about 5 atom % or even more specifically at least about 10 atom %, or yet even more specifically at least about 20 atom %.

In one embodiment, the amount of oxygen on the surface of the porous graphitic carbon apart from that of any heteroatoms of the bonded functional groups is limited. In one embodiment, the atomic weight percent of such oxygen in the stationary phase is less than about 25 atom %, more specifically less than 20 atom % and even more specifically less than about 15 atom %. In one embodiment, the atomic weight percent of functional group atoms other than oxygen is greater than the atom % of oxygen in the stationary phase. In one embodiment, the atomic weight percent of functional group atoms other than oxygen is at least about twice that of the atomic weight percent of oxygen in the stationary phase material. For example, for DTAP, oxygen comprises approximately 23 atom % of the functional group atoms.

The covalent functionalization of the graphitic surface with the one or more alkyl groups and optional heteroatoms is sufficiently extensive to cause an appreciable difference in the separation efficiency of a separation apparatus incorporating the functionalized graphite stationary phase materials as compared to non-functionalized porous graphitic carbon.

### V. SEPARATION APPARATUSES AND METHODS

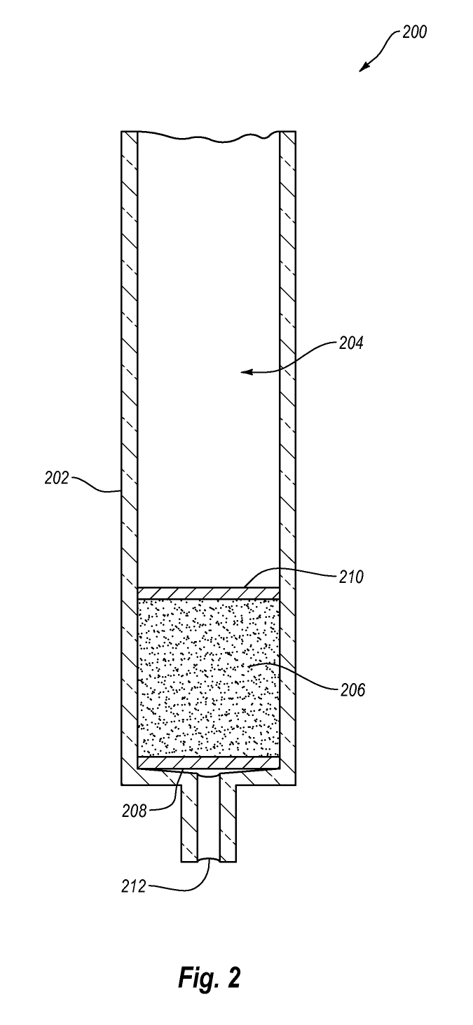

FIG. 2 is a cross-sectional view of a separation apparatus 200 according to an embodiment. The separation apparatus 200 may include a column 202 defining a reservoir 204. A porous body 206 (e.g., a porous composite bed, porous disk, other porous mass, etc.) may be disposed within at least a portion of the reservoir 204 of the column 202. The porous body 206 may comprise any of the functionalized graphitic stationary phase materials disclosed herein. The porous body 206 is porous so that a mobile phase may flow therethrough. In various embodiments, a frit 208 and/or a frit 210 may be disposed in column 202 on either side of porous body 206. The frits 208 and 210 may comprise any suitable material that allows passage of a mobile phase and any solutes present in the mobile phase, while preventing passage of the functionalized graphitic stationary phase materials present in porous body 206. Examples of materials used to form the frits 208 and 210 include, without limitation, glass, polypropylene, polyethylene, stainless steel, polytetrafluoroethylene, or combinations of the foregoing.

The column 202 may comprise any type of column or other device suitable for use in separation processes such as chromatography and/or solid phase extraction processes. Examples of the column 202 include, without limitation, chromatographic and solid phase extraction columns, tubes, syringes, cartridges (e.g., in-line cartridges), and plates containing multiple extraction wells (e.g., 96-well plates). The reservoir 204 may be defined within an interior portion of the column 202. The reservoir 204 may permit passage of various materials, including various solutions and/or solvents used in chromatographic and/or solid-phase extraction processes.

The porous body 206 may be disposed within at least a portion of reservoir 204 of the column 202 so that various solutions and solvents introduced into the column 202 contact at least a portion of the porous body 206. The porous body 206 may comprise a plurality of substantially non-porous particles in addition to the composite porous material.

In certain embodiments, frits, such as glass frits, may be positioned within the reservoir 204 to hold porous body 206 in place, while allowing passage of various materials such as solutions and/or solvents. In some embodiments, a frit may not be necessary, such as where a monolithic functionalized graphitic stationary phase is used.

In one embodiment, the separation apparatus 200 is used to separate two or more components in a mobile phase by causing the mobile phase to flow through the porous body 206. The mobile phase is introduced through an inlet and caused to flow through the porous body 206 and the separated components may be recovered from the outlet 212.

In one embodiment, the mobile phase includes concentrated organic solvents, acids, or bases. In one embodiment, the mobile phase includes a concentrated acid with a pH less than about 3, more specifically less than about 2. In another embodiment, the mobile phase includes a base with a pH greater than about 10, more specifically greater than about 12, and even more particularly greater than about 13.

In one embodiment, the separation apparatus 200 is washed between a plurality of different runs where samples of mixed components are separated. In one embodiment, the washing may be performed with water. In another embodiment, a harsh cleaning solvent may be used. The harsh cleaning solvent may be a concentrated organic solvent and/or a strong acid or base. In one embodiment, the cleaning solvent has a pH less than about 3, more specifically less than about 2. In another embodiment, the cleaning solvent has a pH greater than about 10, more specifically greater than about 12, and even more particularly greater than about 13.

### VI. EXAMPLES

The following examples are for illustrative purposes only and are not meant to be limiting with regards to the scope of the specification or the appended claims.

### Example 1

Example 1 describes the synthesis of a functionalized graphitic stationary phase material using ATB.

The carbon-nitrogen bond of ATB undergoes hemolytic cleavage at elevated temperatures as shown below:

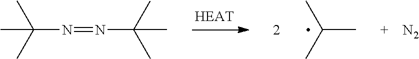

A column was in-house packed with high surface area porous graphite (i.e., HYPERCARB) was obtained from Thermo Fisher. The column dimensions were 4.6 mm ID×50 mm L, and the porous graphite particles had a 5 μm average particle size. The pre-packed HYPERCARB column was interfaced with an HP 5890 Series II GC. The column was dried prior to functionalization by purging the column with N₂ at 50° C. overnight. The injector port of the GC was maintained at 145° C. with the GC oven set at 235° C. The temperature settings were predetermined to cause volatilization and hemolytic cleavage of the ATB. Other temperature settings could be used, so long as the conditions (e.g., temperature and pressure) are sufficient to cause volatilization of the functionalizing agent and radical formation. The radical intermediates react with the porous graphitic carbon, resulting in covalent bonding of tert-butyl groups to the graphitic carbon material. Repeated 25 μL aliquots of ATB were injected to functionalize the graphitic particles. Injections of the ATB functionalizing agent were done every five minutes, which allows the reaction to occur along with allowing the column to be purged of any volatile compounds prior to further injections of the ATB. A total of 0.5 mL of ATB functionalizing agent was injected into the column. After functionalizing the material, the column was interfaced with an LC pump and cleaned with 50 mL of xylenes, 50 mL of a 1:1 v/v xylenes/hexanes mix, and 800 mL of methanol. After cleaning with methanol, the column was ready for LC measurements to cause homolytic cleavage between the carbon-iodine bond, thereby forming a radial intermediate that reacted with the porous graphitic carbon. Twenty aliquots of 25 μL were delivered every 5 minutes over a period of 100 minutes.

Prior to functionalizing the graphitic particles, the retention times (Rₜ), retention factor (k′), and plates/meter characteristics for 12 different analytes were determined as presented in Table I below. The retention factor k′ is calculated as retention time minus tₘ divided by tₘ (the ratio of time an analyte is retained in the stationary phase to the time it is retained in the mobile phase). Ninety percent confidence interval values (90% C.I.) are also recorded for the various characteristics. After functionalization, the same 12 analytes were used to determine if there was any difference in retention times. These results are presented in Table II below. In both cases, the mobile phase was 5% v/v water in methanol at a flow rate of 0.8 mL/min, a temperature of 30.0° C., and the spectral analysis wavelength used was 254 nm. In Table I, the backpressure varied between 541 psi and 565 psi. In Table II, the backpressure varied between 447 psi and 471 psi. The procedure was repeated multiple times and the data obtained were reproducible and indicate that the porous graphitic carbon had been modified, providing evidence that the graphitic carbon had been functionalized with tert-butyl radicals from the ATB.

TABLE I
Retention Time Data Prior to Functionalization

| Analyte | tₘ | Rₜ | 90% C.I. | plates/m | 90% C.I. | k′ | 90% C.I. | Asymmetry 10% |
|---|---|---|---|---|---|---|---|---|
| benzene | 0.932 | 1.184 | ±0.001 | 27978 | ±71 | 0.27 | ±0.001 | 1.45 |
| isopropylbenzene | 0.931 | 1.461 | ±0.001 | 33244 | ±402 | 1.047 | ±0.001 | 1.45 |
| toluene | 0.931 | 1.538 | ±0.001 | 36991 | ±253 | 0.653 | ±0.001 | 1.69 |
| ethylbenzene | 0.929 | 1.563 | ±0.000 | 36808 | ±159 | 0.682 | ±0.002 | 1.61 |
| n-propylbenzene | 0.931 | 1.905 | ±0.001 | 41275 | ±604 | 0.569 | ±0.005 | 1.82 |
| p-isopropyltoluene | 0.930 | 1.925 | ±0.001 | 42526 | ±255 | 1.068 | ±0.003 | 1.63 |
| m-xylene | 0.929 | 2.347 | ±0.002 | 49619 | ±433 | 1.526 | ±0.006 | 1.90 |
| n-butylbenzene | 0.931 | 2.482 | ±0.001 | 46583 | ±172 | 1.666 | ±0.001 | 2.11 |
| p-xylene | 0.929 | 2.599 | ±0.001 | 51224 | ±483 | 1.798 | ±0.002 | 1.96 |
| o-xylene | 0.931 | 4.064 | ±0.000 | 51179 | ±325 | 1.844 | ±0.004 | 2.14 |
| 1,3,5-trimethylbenzene | 0.931 | 4.064 | ±0.004 | 64096 | ±416 | 3.367 | ±0.006 | 1.96 |
| phenyl hexane | 0.928 | 5.757 | ±0.003 | 48763 | ±355 | 5.202 | ±0.008 | 3.17 |

TABLE II
Retention Time Data After Functionalization

| Analyte | tₘ | Rₜ | 90% C.I. | plates/m | 90% C.I. | k′ | 90% C.I. | Asymmetry 10% |
|---|---|---|---|---|---|---|---|---|
| benzene | 0.932 | 1.186 | ±0.001 | 26226 | ±244 | 0.272 | ±0.001 | 1.635 |
| isopropylbenzene | 0.931 | 1.487 | ±0.000 | 30654 | ±126 | 0.597 | ±0.000 | 1.756 |
| toluene | 0.931 | 1.561 | ±0.000 | 33143 | ±146 | 0.676 | ±0.001 | 1.989 |
| ethylbenzene | 0.932 | 1.587 | ±0.001 | 33511 | ±194 | 0.702 | ±0.002 | 1.894 |
| n-propylbenzene | 0.930 | 1.967 | ±0.001 | 35631 | ±113 | 1.116 | ±0.000 | 2.319 |
| p-isopropyltoluene | 0.930 | 2.014 | ±0.001 | 38405 | ±357 | 1.164 | ±0.001 | 2.008 |
| m-xylene | 0.931 | 2.421 | ±0.001 | 41704 | ±299 | 1.599 | ±0.002 | 2.465 |
| n-butylbenzene | 0.932 | 2.604 | ±0.001 | 36868 | ±773 | 1.795 | ±0.002 | 2.924 |
| p-xylene | 0.932 | 2.785 | ±0.001 | 40004 | ±3514 | 1.989 | ±0.002 | 2.783 |
| o-xylene | 0.931 | 2.709 | ±0.001 | 44028 | ±131 | 1.908 | ±0.002 | 2.684 |
| 1,3,5-trimethylbenzene | 0.930 | 4.406 | ±0.001 | 52718 | ±353 | 3.738 | ±0.006 | 2.390 |
| phenyl hexane | 0.931 | 6.544 | ±0.003 | 17286 | ±195 | 6.032 | ±0.002 | 5.057 |

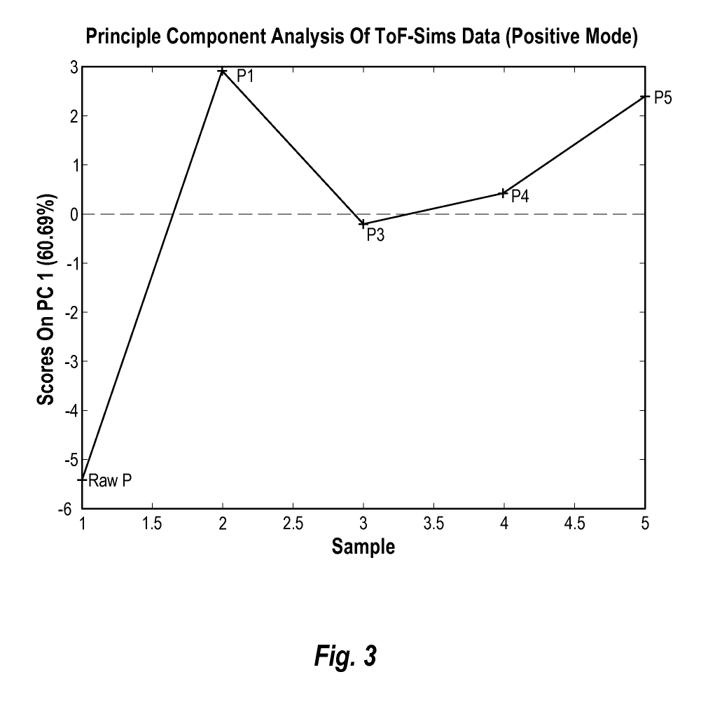

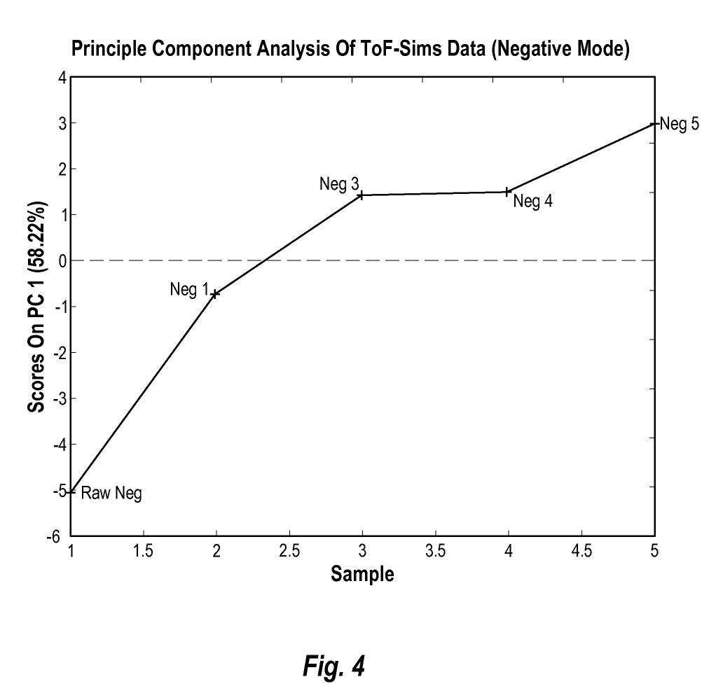

The reacted graphite sample was characterized by ToF-SIMS. Principle component analysis (PCA) of the ToF-SIMS spectra for Example 1 is shown in FIGS. 3-4. The principle component analysis shows that there is a statistical difference in the ToF-SIMS data for the unfunctionalized graphitic carbon material (Raw P in FIG. 3 and Raw Neg in FIG. 4) as compared to the functionalized material, providing evidence that the graphitic carbon material has been functionalized by covalent bonding of a functional group onto the graphitic carbon. P1, P3, P4, and P5 correlate to the data of the functionalized graphitic carbon material for positive mode ToF-SIMS analysis, and Neg 1, Neg 3, Neg 4, and Neg 5 correlate to the data of the functionalized graphitic carbon material for negative mode ToF-SIMS analysis.

### Example 2

Example 2 is a prophetic example describing the synthesis of a functionalized graphitic stationary phase material using DTAP.

The carbon-nitrogen bond of DTAP undergoes hemolytic cleavage at elevated temperatures as shown below:

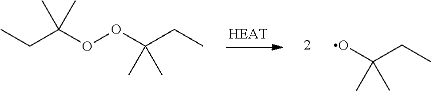

Functionalization and testing is carried out in a similar manner as described above with respect to Example 1.

Chromatograms of functionalized material are expected to provide longer retention times and less tailing of the chromatographic peaks (i.e., better symmetry) as compared to where the functionalization is carried out in the liquid/solution phase. Examples and additional details of functionalization in the liquid/solution phase are disclosed in U.S. patent application Ser. No. 12/563,646, entitled “FUNCTIONALIZED GRAPHITIC STATIONARY PHASE AND METHODS FOR MAKING AND USING SAME” filed 21 Sep. 2009, which is incorporated herein, in its entirety, by this reference.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

What is claimed is:

1. A gas-phase method for preparing a functionalized graphitic stationary phase material suitable for use in a separation apparatus, comprising:
providing porous graphitic carbon having a porosity and surface area suitable for use as a stationary phase;
volatilizing a functionalizing agent so that the functionalizing agent is in a gas-phase; and
functionalizing at least a portion of the surface area of the porous graphitic carbon by:
forming a radical from the gas-phase functionalizing agent; and
covalently bonding the radical to the porous graphitic carbon to yield the functionalized graphitic stationary phase material.

2. The method of claim 1, wherein the porous graphitic carbon comprises a plurality of graphitic particles exhibiting an average particle size of at least about 1 μm and a surface area per unit weight of at least about 5.0 m²/g.

3. The method of claim 1, wherein the functionalizing agent comprises at least one member selected from the group consisting of alkyl halides, azo compounds, benzoyl peroxide, diacyl peroxides, alkyl peroxy acids, dialkyl peroxides, tri-peroxides, peroxyesters, perfluoronated peroxides, tertiary alcohols, hydroperoxides, molecules with two or more double bonds, and molecules with epoxide groups.

4. The method of claim 3, wherein the at least one member is an azo compound that includes azo-tert-butane.

5. The method of claim 3, wherein the at least one member is a dialkyl peroxide that includes di-tert-amylperoxide.

6. The method of claim 3, wherein the at least one member is a dialkyl peroxide in combination with a tertiary alcohol.

7. The method of claim 6, wherein the tertiary alcohol comprises an alkyl group having 18 carbon atoms.

8. The method as in claim 1, wherein forming a radical from the functionalizing agent comprises at least one of heating the functionalizing agent or exposing the functionalizing agent to a wavelength of light selected to cleave the functionalizing agent and form a radical.

9. The method as in claim 8, wherein cleavage of the functionalizing agent is homolytic so as to result in two identical radicals.

10. The method as in claim 1, wherein volatilizing a functionalizing agent so that the functionalizing agent is in a gas-phase comprises heating the functionalizing agent in the presence of the porous graphitic carbon.

11. The method as in claim 10, wherein the gas-phase functionalizing agent and the porous graphitic carbon are heated to a temperature between about 100° C. and about 300° C. during volatilization of the functionalizing agent.

12. The method as in claim 1, wherein the preparation of the functionalized graphitic stationary phase material is within a chromatography column.

13. The method as in claim 12, wherein the functionalizing agent is introduced non-continuously, at repeated intervals into the graphitic stationary phase material within the chromatography column.

14. The method as in claim 13, wherein the functionalizing agent is introduced over a period of not more than about 2 hours.

15. The method as in claim 1, wherein the preparation of the functionalized graphitic stationary phase material comprises agitating the graphitic stationary phase during functionalization.

16. The method as in claim 15, wherein the functionalizing agent is introduced non-continuously at repeated intervals into the graphitic stationary phase material.

17. The method as in claim 16, wherein the functionalizing agent is introduced over a period of not more than about 2 hours.

18. A functionalized graphitic stationary phase for use in separation apparatus, comprising:
porous graphitic carbon having a porosity and surface area suitable for use as a stationary phase in a separation apparatus; and
a plurality of functional group molecules covalently bonded to the surface of the porous graphitic carbon, the functional group molecules comprising at least one alkyl group selected from the group consisting of a tert butyl group and an alkyl group terminated by an oxygen heteroatom.

19. The functionalized graphitic stationary phase as in claim 18, wherein the alkyl groups comprises an amyl group.

20. A separation apparatus, comprising:
a vessel having an inlet and an outlet; and
the functionalized graphitic stationary phase according to claim 18 packed within the vessel.
